(19) United States
(12) Patent Application Publication
(10) Pub. No.: US 2019/0151157 A1
(43) Pub. Date: May 23, 2019

(54) STRAPPED BANDAGE

(71) Applicant: Dru-Kare LLC, Cottage Grove, MN (US)

(72) Inventors: Karin M. Herder, Cottage Grove, MN (US); Drew J. Sanders, Cottage Grove, MN (US)

(21) Appl. No.: 15/891,595

(22) Filed: Feb. 8, 2018

Related U.S. Application Data
(63) Continuation-in-part of application No. 29/626,957, filed on Nov. 21, 2017.

(51) Int. Cl. A61F 13/00; A61F 7/08

(52) U.S. Cl. CPC A61F 13/00085; A61F 13/00042; A61F 2013/00187; A61F 7/08; A61F 13/00063

(57) ABSTRACT

Embodiments of strapped bandages for treating wounds on a subject patient are disclosed, comprising a pocket for receiving a heating and/or cooling packet therein.

### CROSS-REFERENCE TO RELATED APPLICATIONS

This application claims priority to design patent application Ser. No. 29/626,957, filed Nov. 21, 2017 and titled STRAPPED BANDAGE WITH HEATING/COOLING POCKET and to design patent application Ser. No. 29/626,965, filed Nov. 21, 2017 and titled STRAPPED BANDAGE, the entire contents of which are incorporated herein by reference.

### STATEMENT REGARDING FEDERALLY SPONSORED RESEARCH OR DEVELOPMENT

Not Applicable

### INCORPORATION BY REFERENCE

All references, including but not limited to publications, patent applications and patents mentioned in this specification are hereby incorporated by reference to the same extent and with the same effect as if each reference was specifically and individually indicated to be incorporated by reference.

### FIELD OF THE INVENTION

The inventions described herein relate to bandages for patients. More specifically, bandages comprising adjustable straps. Still more specifically, bandages comprising a pocket for receiving a heating or cooling packet therein.

### BACKGROUND OF THE INVENTION

Bandages for dressing wounds are known in the art. However, known bandages require securing against a wound by an adhesive integrated into a portion of the bandage or with adhesive tape. In addition, known bandages do not have the ability to incorporate a heating and/or cooling packet within the bandage. Various embodiments of the several inventions disclosed herein address these, inter alia, issues.

Applicant has filed design patent application Ser. No. 29/626,965 on Nov. 21, 2017 entitled STRAPPED BANDAGE, and design patent application Ser. No. 29/626,957 on Nov. 21, 2017 entitled STRAPPED BANDAGE WITH HEATING AND COOLING POCKET, the entire contents of which are hereby incorporated by reference.

### BRIEF DESCRIPTION OF THE SEVERAL VIEWS OF THE DRAWINGS

FIG. 1 illustrates a perspective view of one embodiment of the present invention.

FIG. 2 illustrates a top view of one embodiment of the present invention.

FIG. 3 illustrates a side view of one embodiment of the present invention.

FIG. 4 illustrates a bottom view of one embodiment of the present invention.

FIG. 5 illustrates an end view of one embodiment of the present invention.

FIG. 6 illustrates a perspective view of one embodiment of the present invention.

FIG. 7 illustrates a perspective view of one embodiment of the present invention in combination with a heating/cooling pack.

FIG. 8 illustrates a perspective view of one embodiment of the present invention with a heating/cooling pack inserted.

FIG. 9 illustrates a cross-sectional view of the embodiment of the present invention shown in FIG. 8.

FIG. 10 illustrates one embodiment of an embodiment of the present invention attached to an arm of a subject patient.

### DETAILED DESCRIPTION OF THE INVENTION

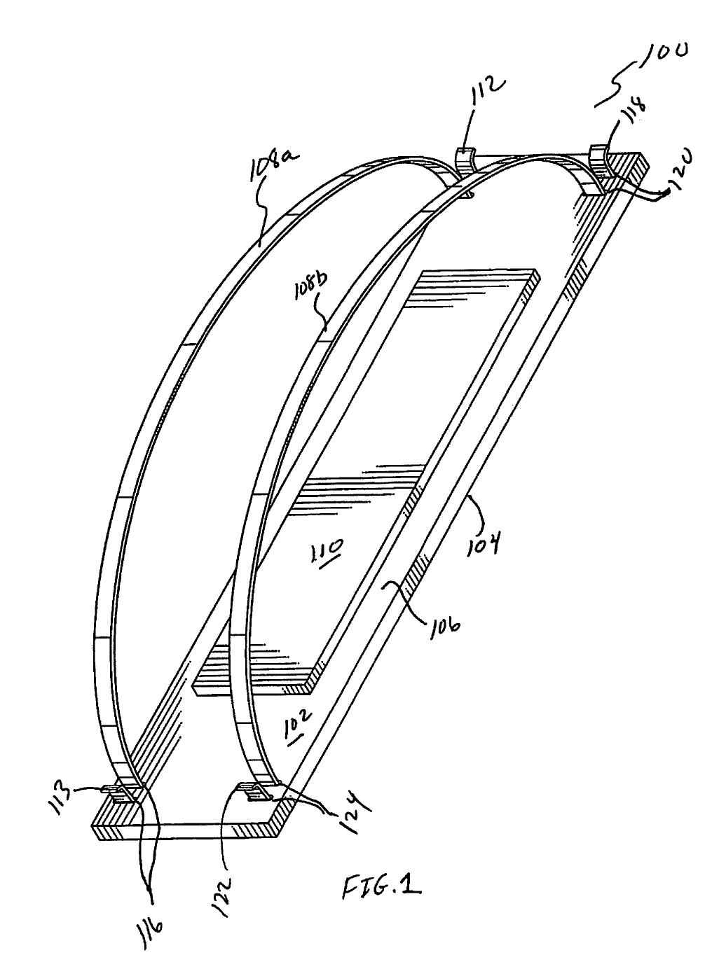

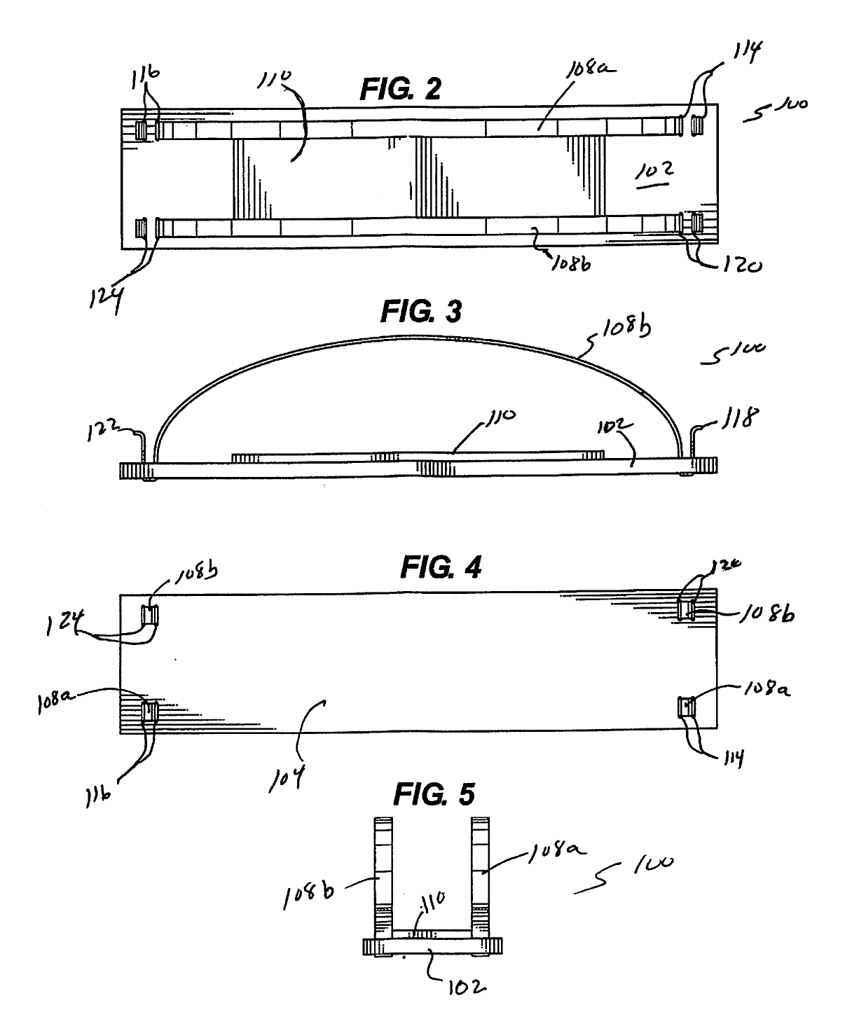

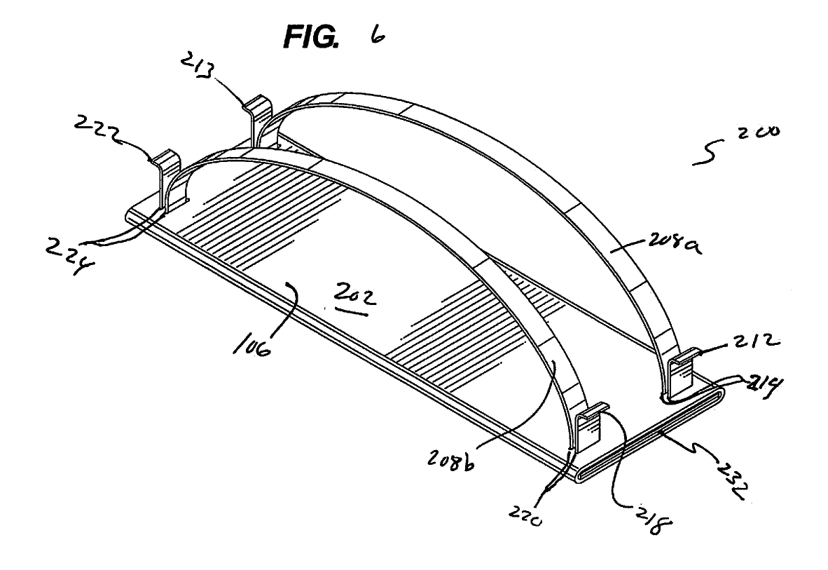

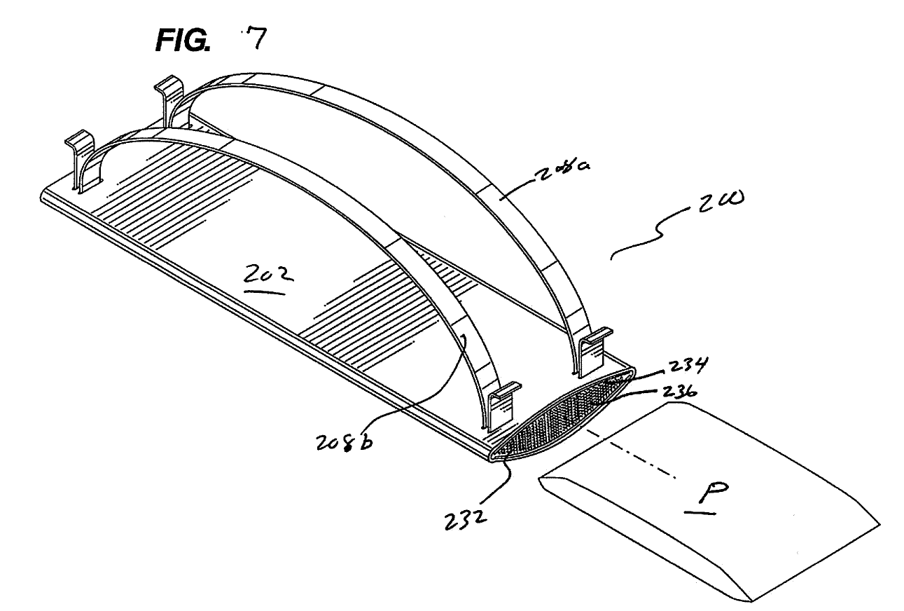

FIGS. 1-5 illustrates one embodiment of a strapped bandage 100 comprising a base 102 with an upper surface 104, a lower surface 106, two straps 108a, 108b adjustable attached to the base 102 and an absorbent section 110 attached to the lower surface 106. Straps 108a, 108b are arranged so that a subject patient may slip the straps 108a, 108b over an appendage, e.g., an arm or a leg, or over the head such that the lower surface 106 and the absorbent section 110 are arranged to contact the patient's body and the wound thereon. This arrangement is best illustrated in FIG. 10.

A first strap 108a comprises a first end 112 extending through a first set of holes 114 in the base 102, and a second end 113 extending through a second set of holes 116 in the base 102. Second strap 108b comprises a first end 118 extending through a third set of holes 120 in the base 102 and a second end 122 extending through a fourth set of holes 124 in the base 102.

The strapped bandage 100 may be adjusted to accommodate sizing of various bodily locations. The portion of the strap 108a located between the first and second sets of holes 114, 116 may be shortened by pulling the first end 112 and/or the second end 113 through holes 114 and/or 116. The portion of the strap 108a located between the first and second sets of holes 114, 116 may be lengthened by pulling on the strap portion located between first and second sets of holes 114, 116.

Similarly, the portion of the strap 108b located between the third and fourth sets of holes 120, 124 may be shortened by pulling the first end 118 and/or the second end 122 through holes 120 and/or 124. The portion of the strap 108b located between the third and fourth sets of holes 120, 124 may be lengthened by pulling on the strap portion located between third and fourth sets of holes 120, 124.

Absorbent section 110 may comprise a gauze material or similar material and may be pre-loaded with a medication, e.g., an antibiotic. Absorbent section 110 may be removably affixed to base 102 to permit removal of absorbent section 110 and removable attachment of a fresh absorbent section 110 thereto to facilitate changing the dressing while retaining the remainder of the bandage 100.

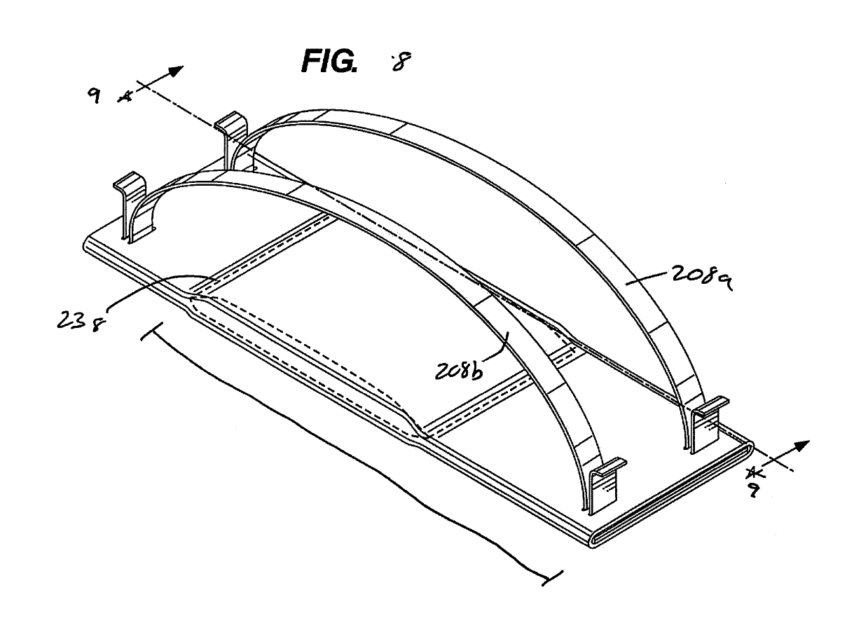

Turning now to FIGS. 6-10, another form of a strapped bandage 200 is provided.

Strapped bandage 200 is similar in certain respects to the embodiments illustrated in FIGS. 1-5. Therefore, bandage 200 comprises a base 202 with an upper surface 204, a lower surface 206, two straps 208a, 208b adjustable attached to the base 202. Straps 208a, 208b are arranged so that a subject patient may slip the straps 208a, 208b over an appendage, e.g., an arm or a leg, or over the head as best shown in FIG. 10.

A first strap 208a comprises a first end 212 extending through a first set of holes 214 in the base 202, and a second end 213 extending through a second set of holes 216 in the base 202. Second strap 208b comprises a first end 218 extending through a third set of holes 220 in the base 202 and a second end 222 extending through a fourth set of holes 224 in the base 202.

The strapped bandage 200 may be adjusted to accommodate sizing of various bodily locations. The portion of the strap 208a located between the first and second sets of holes 214, 216 may be shortened by pulling the first end 212 and/or the second end 213 through holes 214 and/or 216. The portion of the strap 208a located between the first and second sets of holes 214, 216 may be lengthened by pulling on the strap portion located between first and second sets of holes 214, 216.

Similarly, the portion of the strap 208b located between the third and fourth sets of holes 220, 224 may be shortened by pulling the first end 218 and/or the second end 222 through holes 220 and/or 224. The portion of the strap 208b located between the third and fourth sets of holes 220, 224 may be lengthened by pulling on the strap portion located between third and fourth sets of holes 220, 224.

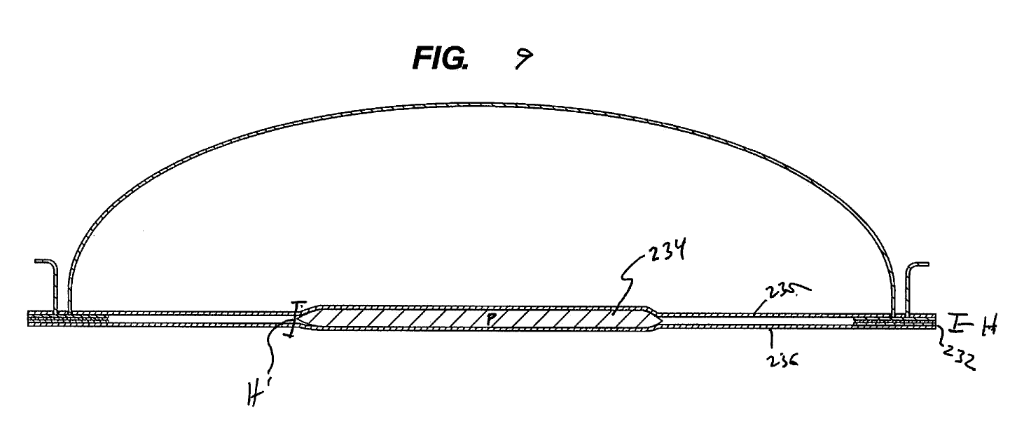

As best shown in FIGS. 7 and 8, a pocket 234 is defined within the base 202 sized to accommodate receiving of a heating and/or cooling packet P therein for subsequent strapped secured application to a wounded area of a subject patient. Pocket 234 may be defined by an opening 232 in base 202 further defined by a first interior side 235 and a second interior side 236 within the base 202, the interior sides forming and defining the pocket 234. Pocket 234 may further comprise a length that is defined by the substantially equal lengths of the first interior side 235 and the second interior side 236, wherein the first and second interior sides 235, 236 comprise a common end 238 which fixes the endpoint of the pocket 234 within base 202. The common end 238 thus defines the furthest distance that a heating and/or cooling pocket may be inserted into the pocket 234 from the opening 232.

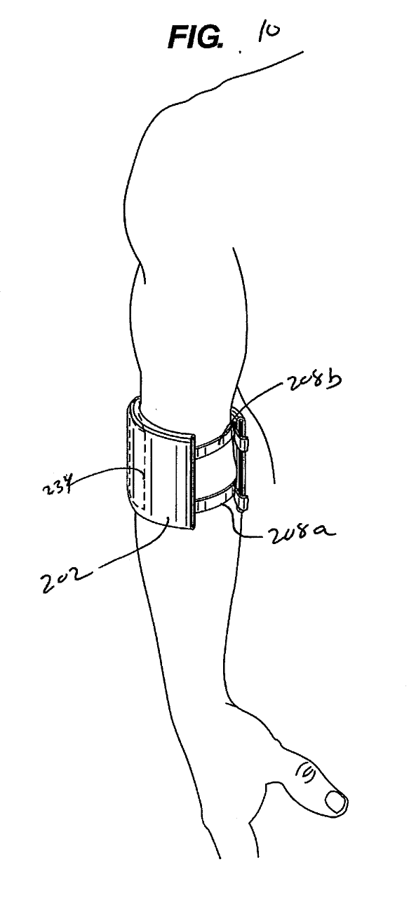

Alternatively as seen in both FIGS. 8 and 9, the pocket 234 as defined and formed by the first and second interior sides 235, 236 may define a region of greater height H′ as compared with the height H defined between first and second interior sides 235, 236 proximate opening 232, wherein H is less than H′. The region of greater height H′ may thus accommodate the heating and/or cooling packet P, holding it in place within the region of great height H′. In this embodiment, the first and second interior sides 235, 236 in the region of lesser height H may be made of a fabric or material that stretches to allow insertion of the heating and/or cooling packet P wherein the dimensions of the packet P are slightly larger than the region of lesser height H.

FIG. 10 shows one embodiment of strapped bandage 200 attached and secured to a subject patient's arm with a heating and/or cooling packet P inserted within pocket 234

As will be readily understood, the heating and/or cooling packet may be removed when its utility has ended and, when necessary, a new heating and/or cooling packet may be inserted into the pocket.

The description of the various inventions, embodiments thereof and applications as set forth herein is illustrative and is not intended to limit the scope of the invention. Features of various embodiments may be combined with other embodiments within the contemplation of these inventions. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the inventions.

What is claimed is:

1. A method of treating a wound on a patient with a strapped bandage with heating and/or cooling, comprising:
providing a bandage with two adjustable straps, a base, the base defining an opening on one end and a pocket within the base, the opening and pocket adapted to receive a heating and cooling packet therein;
inserting the heating and/or cooling packet into the opening and pocket;
securing the bandage to the patient at a location over the wound with the adjustable straps;
removing the heating and/or cooling packet from the pocket; and
inserting another heating and/or cooling packet into the pocket.

2. A strapped bandage comprising:
a base;
two adjustable straps disposed through the base;
a pocket defined within the base and accessible through an opening in the base.

3. The strapped bandage of claim 2, wherein the pocket and the opening in the base each comprise a height, the height of the pocket and the opening in the base being substantially equivalent.

4. The strapped bandage of claim 2, wherein the pocket and the opening in the base comprise a height, the height of the pocket being greater than the height of the opening in the base.

5. The strapped bandage of claim 2, wherein the opening and the pocket are defined by first and second interior sides.

6. The strapped bandage of claim 5, wherein the first and second interior sides comprise a common endpoint.
